(19) United States
(12) Patent Application Publication
(10) Pub. No.: US 2009/0028402 A1
(43) Pub. Date: Jan. 29, 2009

(54) Medical Image System

(76) Inventor: Takanori Ando, Tokyo (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/224,105

(22) PCT Filed: Feb. 20, 2007
(86) PCT No.: PCT/JP2007/053069

§ 371 Date: Aug. 18, 2008

(30) Foreign Application Priority Data
Feb. 28, 2006 (JP) 2006-052155

(51) Int. Cl. A61B 5/00

(52) U.S. Cl. 382/128

(57) ABSTRACT

A medical image system capable of performing display corresponding to different monitor configurations of medical image display apparatuses is provided. In the medical image system (1) including the medical image display apparatuses (5A, 5B) having one or more monitors, a server (3) stores display format files of object data corresponding to the respective monitor configurations in the plurality of medical image display apparatuses (5A, 5B) and discriminates the monitor configuration in the medical image display apparatus (5A or 5B) for performing display of the object data in the medical image display apparatuses (5A, 5B). Then, in the stored display format files, the display format file corresponding to the discriminated monitor configuration is transmitted to the medical image display apparatus (5A or 5B). The medical image display apparatus (5A or 5B) displays the object data on monitor(s) based on the transmitted display format file.

### TECHNICAL FIELD

The present invention relates to a medical image system for displaying medical image data and display object data for other medical images on a monitor.

### BACKGROUND ART

In recent years, in hospitals, digitalizing for electronic data of medical image is advanced, and medical image systems for series of processes from photography of the medical image to displaying are constructed. This is used to store data of a medical images obtained by a photographic apparatus, and so on in a bulk memory, manage it by a server, and distribute it to medical image display apparatuses installed at each area such as a consultation room, and so on in a hospital.

When performing radiogram interpretation of a medical image, since the doctor arranges and checks a plurality of medical images and may cause to display it in parallel a patient list screen or a report creation screen, and so on together with the medical image, many medical image display apparatuses have not only one but a plurality of monitors.

Conventionally, since it is complicated to set which monitor should display what display contents every time the medical image display apparatus is used, the display format is prepared for each doctor and stored in a server, the display format corresponding to a login doctor is fetched from the server in a case of login to the display apparatus, and display is performed based on it (see e.g. Patent document 1).

[Patent document 1] Japanese Patent Laid-Open No. 11-282936

### DISCLOSURE OF THE INVENTION

### The Problem to be Solved by the Invention

However, the doctor may not use the same medical image display apparatus always, the doctor may do radiogram interpretation after login to a medical image display apparatus set at a different location. By the conventional method, it is premised that a doctor uses one some medical image display apparatus as fixed action and one display format for each doctor is set. Accordingly, when a display apparatus of a different monitor configuration is used, the display is performed by a display format that does not match the monitor configuration of the display apparatus.

For example, when it is premised that the configuration of three monitors is used, and the doctor logged in to a display apparatus of the configuration of one monitor in spite of setup for a display format for displaying a patient list screen, a radiogram interpretation screen (display of medical image), and a report screen on each monitor, it becomes the display mode in which three screens are displayed in a overlapped status because the abovementioned display format can not be adapted, and thus the display mode becomes a mode which is not anticipated by the doctor.

It is an object of the present invention to provide a medical image system capable of performing display corresponding to different monitor configurations of medical image display apparatuses.

### Means of Solving the Problem

The present invention of claim 1 is characterized in that

a medical image system including a plurality of medical image display apparatuses each equipped with one or more monitors, comprises:

a storage section for storing a display format of object data corresponding to monitor configuration in each of the plurality of medical image display apparatuses;

a discriminating section for discriminating the monitor configuration in the medical image display apparatus that displays the object data from among the plurality of medical image display apparatuses; and

a display control section that causes a monitor of the medical image display apparatus to display the object data based on the display format corresponding to the discriminated monitor configuration from among the stored display formats.

### EFFECT OF THE INVENTION

According to the invention of claims 1 and 3, it is possible to display object data in the display format corresponding to the monitor configuration even when logged in to any one of medical image display apparatuses having different monitor configurations. Accordingly, the user does not need to perform a display operation one by one to match the monitor configuration of the medical image display apparatus to which the user logged in, and the operability is improved.

According to the invention of claim 2, it not only can respond to the monitor configuration, but it can display the object data by the display format for the user. Thereby, it is possible to perform customization of the display contents, and convenience is improved.

According to the invention of claim 4, it is possible to set the object data to be displayed for each monitor according to the monitor configuration of the medical image display apparatus, and further perform detailed customization of the display contents.

According to the invention of claim 5, it is possible to avoid the overlap display of a plurality of object data in one monitor by lack of monitor.

### BRIEF DESCRIPTION OF DRAWINGS

FIG. 1 shows a diagram representing configuration of a medical image system in the embodiment.

FIG. 2 shows a diagram representing configuration of a server in FIG. 1.

FIG. 3 shows a figure representing an example of a registration user file stored in a storage section in FIG. 2.

FIG. 4 shows a figure representing an example of a display format table stored in a storage section in FIG. 2.

FIG. 5 shows a figure representing an example of a monitor file stored in a storage section in FIG. 2.

FIG. 6 shows a diagram representing configuration of a medical image display apparatus in FIG. 1.

FIG. 7 shows a flowchart indicating display format file providing processes performed by a server.

FIG. 8 shows a flowchart indicating object data display processes performed by a medical image display apparatus.

FIG. 9 shows a diagram representing a display example of object data.

### EXPLANATION OF REFERENCE NUMERAL

1 medical image system
3 server
31 control section
34 storage section
341 registration user file
342 display format table
343 monitor file
4 NAS
5A, 5B medical image display apparatus
51 control section
53a, 53b, 53c monitor

### BEST MODE FOR CARRYING OUT OF THE INVENTION

First, the configuration is explained.

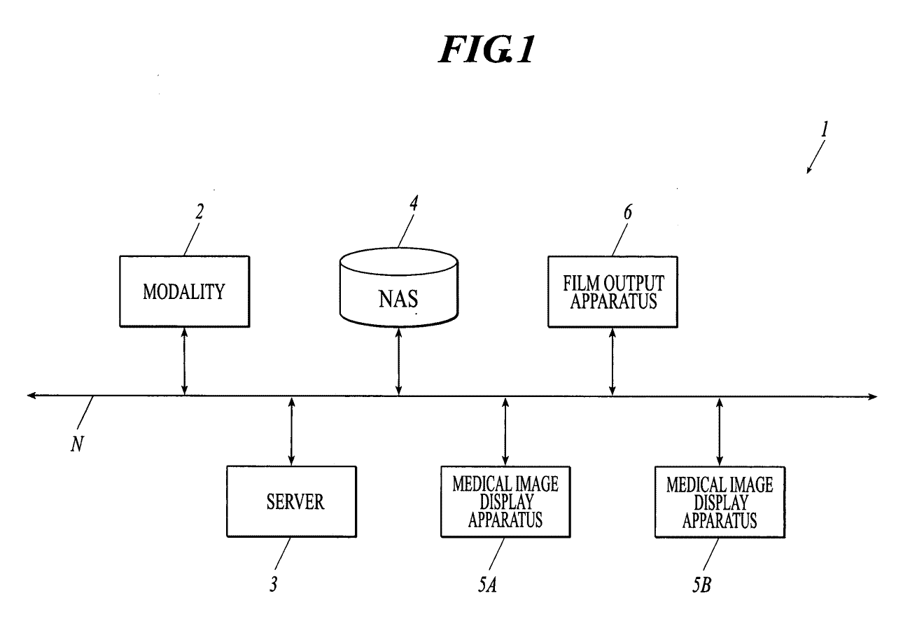

FIG. 1 shows the system configuration of a medical image system 1 in this embodiment.

As shown in FIG. 1, a medical image system 1 is composed of a modality 2, a server 3, a NAS (Network Attached Storage) 4, medical image display apparatuses 5A and 5B, a film output apparatus 6. Each component is connected via network N, The network N is a LAN (Local Area Network) complying with DICOM (Digital Imaging and Communication in Medicine) standard.

The medical image system 1 stores data such as medical images photographed by the modality 2, report, and so on in the NAS 4, and manages its input and output by the server 3. By the control of the server 3, each data stored in the NAS 4 is outputted to the film output apparatus 6 or the medical image display apparatuses 5A and 5B.

Hereinafter, each component will be described.

The modality 2 is an image generation equipment for taking a photograph of patient and generating photographed images (medical images). The various things such as an x-ray equipment, a CT (Computed Tomography) photographic equipment, a MRI (Magnetic Resonance Imaging) photographic equipment, an ultrasonic wave/endoscope photographic equipment, and so on are listed as modality 2. The photographed medical image is transmitted to the NAS 4. In addition, in the modality 2, it is possible to add information relating to the medical image obtained when photographing to the medical image. For example, as the information relating to the medical image, patient information such as patient's name, age, and so on, photography information such as shooting date, modality name, photographing condition, and so on, or image detail information such as bit information of a medical image, and so on are listed. These information may be obtained by the input by cameraman or may be obtained by one inputted from an external device

The server 3 performs overall management of various kinds of data stored in the NAS 4.

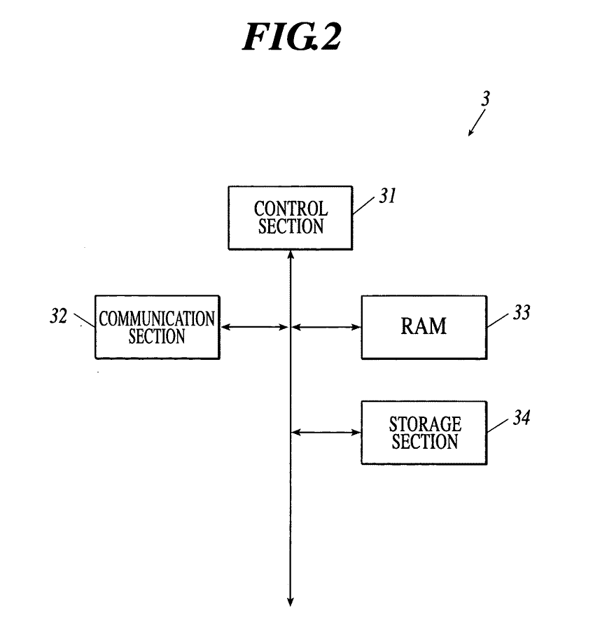

FIG. 2 shows the configuration of the server 3.

As shown in FIG. 2, the server 3 is composed of a control section 31, a communication section 32, a RAM (Random Access Memory) 33, a storage section 34, and so on.

The control section 31 is a CPU (Central Processing Unit), and so on, develops various control programs stored in the storage section 34, and performs operations in cooperation with the program or performs a centralized control of each of sections 32-34.

The communication section 32 is composed of a communication interface such as a network interface card, and so on, and communicates with external devices on the network N. For example, the communication section 32 receives login information of a user from the medical image display apparatuses 5A and 5B, and sends the reply of the result of the user authentication.

The RAM 33 is a working memory to be used by the control section 31.

The storage section 34 stores the various control programs used in the control section 31, parameters or data, and so on necessary for performing the program.

For example, the storage section 34 stores a registration user file 341, a display format table 342, and a monitor file 343.

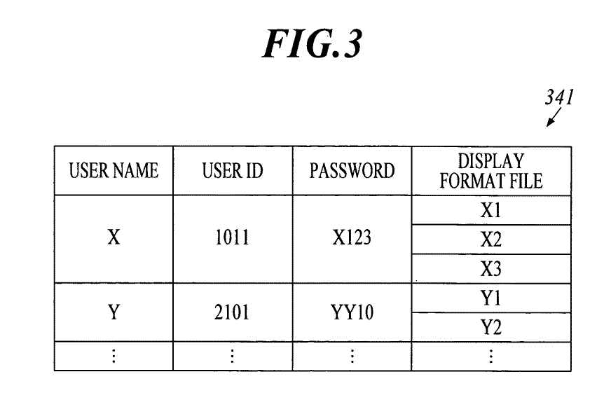

The registration user file 341 is a file for storing user information for matching to perform user authentication. As shown in FIG. 3, the registration user file 341 stores passwords and display format file names by corresponding to the user name, and user ID (number in advance assigned to discriminate users individually). The display format file is a file for setting the display format of data (hereinafter, referred to as object data) of various kinds of display objects, and is prepared for each user. Also, for one user, it is possible to generate a plurality of display format files for the respective monitor configurations or by changing a format of display content. Here, the monitor configuration indicates the number of one or more monitors provided in the medical image display apparatuses 5A and 5B, combinations of various kinds of monitors, and so on.

For example, the user “X” has user ID “1011” and password “X 123”, and it is set that the display format files corresponding to the user “X” are three files of “X1”, “X2”, and “X3”.

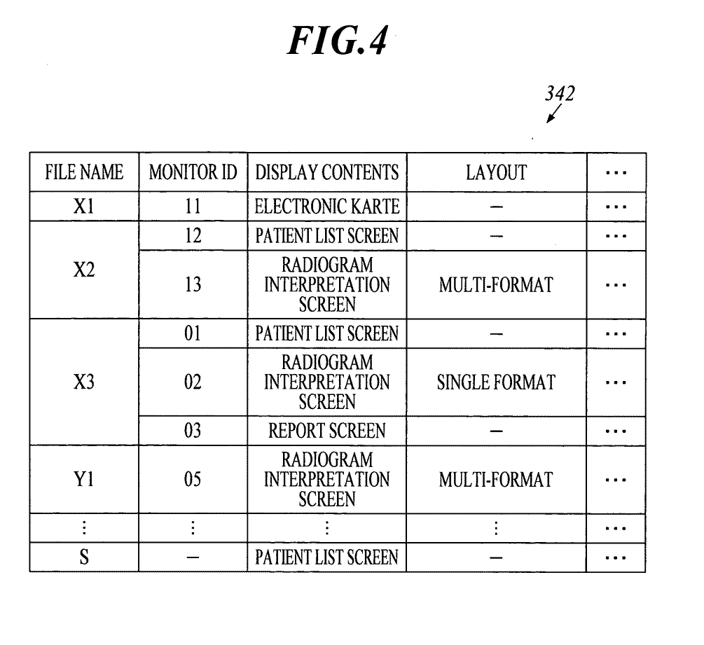

The display format table 342 registers the display format file for each user. As shown in FIG. 4, the display format table 342 stores the file names of the display format files, monitor ID (number assigned in advance to discriminate each monitor individually. MAC address, and so on may be utilized) to be used to display, display contents (kinds of object data) to be displayed on the monitor(s), and each item information of that layout. In addition, though FIG. 4 shows screen names as display contents, a kind of object data to be displayed is set such as patient list data in a patient list screen, medical image data radiogram in an interpretation screen, and report data in a report screen.

For example, the display format file of the file name “X3” indicates the display format in three monitors of monitor ID “01”, “02” and “03”, it is set that the monitor of monitor ID “01” displays “patient list screen”, the monitor of monitor ID “02” displays “radiogram interpretation screen” by layout of “single format”, and the monitor of monitor ID “03” displays “report screen”.

Here, the single format and the multi-format are display formats of medical images. The single format is a format for displaying only one medical image in a display area, and the multi-format is a format for displaying a plurality of medical image in areas to which the display area is divided.

In addition, in the display format table 342, a basic display format file (file name “S”) is registered in addition to the display format file for each user. This is prepared in advance as a standard display format, and the number of monitors for display is set to the minimum “1”. Also, monitor ID is not registered especially to adapt to any monitors, the “patient list screen” necessary for an initial operation is registered as display contents.

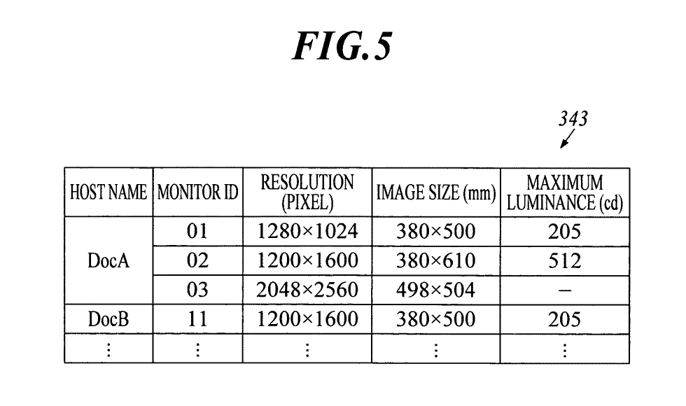

The monitor file 343 is a file for managing information relating to the monitors which all medical image display apparatuses 5A and 5B included in the medical image system 1 have. As shown in FIG. 5, in the monitor file 343, each item information of a resolution, an image size, a maximum luminance of the monitor is stored by corresponding to a host name of the medical image display apparatuses 5A and 5B and monitor ID of the monitors which the medical image display apparatuses 5A and 5B have.

For example, the medical image display apparatus 5A of the host name “DocA” has three monitors of the monitor ID “01”, “02” and “03”, it is set that the monitor of the monitor ID “01” has a resolution “1280×1024 (pixels)”, the image size “380×500” (mm) and the maximum luminance “205” (cd).

The NAS 4 is composed of a bulk memory, and stores various kinds of data relating to medical images such as report, and so on in addition to the medical image. The NAS 4 outputs data instructed according to control of the server 3 to a designated external device.

The medical image display apparatuses 5A and 5B are used at the time of a radiogram interpretation of medical images by a doctor or at the time of medical examination. The medical image display apparatuses 5A and 5B are composed of one or more monitors and displays object data on the monitor(s).

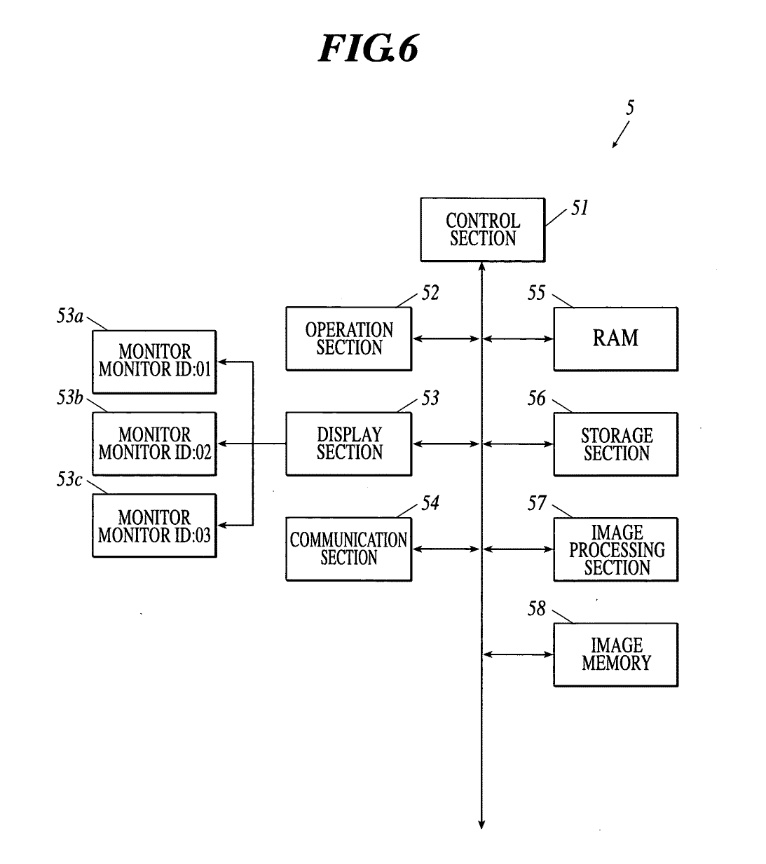

The configuration of the medical image display apparatuses 5A and 5B will be described in view of FIG. 6. Here, however, since the medical image display apparatuses 5A and 5B are same about the fundamental configuration, the medical image display apparatus 5A will be described as a representative.

As shown in FIG. 6, the medical image display apparatus 5A is composed of a control section 51, an operation section 52, a display section 53, a communication sections 54, a RAM 55, a storage section 56, an image processing section 57, and an image memory 58.

The control section 51 is composed of a CPU, and so on, reads the various control programs stored in the storage section 56, develops it into RAM 55, performs the various operations in cooperation with the control programs, and performs a centralized control of each of sections 52-58.

The operation section 52 is composed of a keyboard or mouse, and so on, when these are operated, generates control signals corresponding to the operations, and output it to the control section 51.

The display section 53 is composed of monitors 53a-53c, displays medical image(s) on these monitors 53a-53c, and displays various kinds of object data such as an operation screen, and so on of the report creation screen, the electronic Karte screen, and so on.

As above mentioned, monitor IDs are assigned to these monitor 53a-53c individually, the information of the host name and the monitor ID are stored in advance in the server 3 and managed in the monitor file 343. Here, it is set that the monitor ID of monitor 53a is “01”, the monitor ID of monitor 53b is “02”, and the monitor ID of monitor 53c is “03”.

The communication section 54 has a communication interface, and communicates with an external device on the network N. For example, the communication section 54 transmits request information for requesting object data such as medical image(s), and so on to the server 3, and receives the object data from the NAS 4.

The RAM 55 is a working memory used by the control section 51.

The storage section 56 stores parameters, data, and so on necessary for performing the programs in addition to the various kinds of control programs performed by the control section 51.

The image processing section 57 performs the various kinds of image processes such as reduction, expansion, rotation, reversal, and so on to the object data.

The image memory 58 stores the object data.

The film output apparatus 6 outputs the medical images to a film.

Next, the operations of the medical image system 1 will be described.

In the medical image system 1, a doctor registers display format(s) corresponding to the monitor configuration in the server 3 in advance, and prepares the display format file(s). Even when the doctor logs in to any medical image display apparatus 5A or 5B, it is possible to obtain, from the server 3, the display format file corresponding to the monitor configuration of the medical image display apparatus 5A or 5B to which the login is performed, and utilize it.

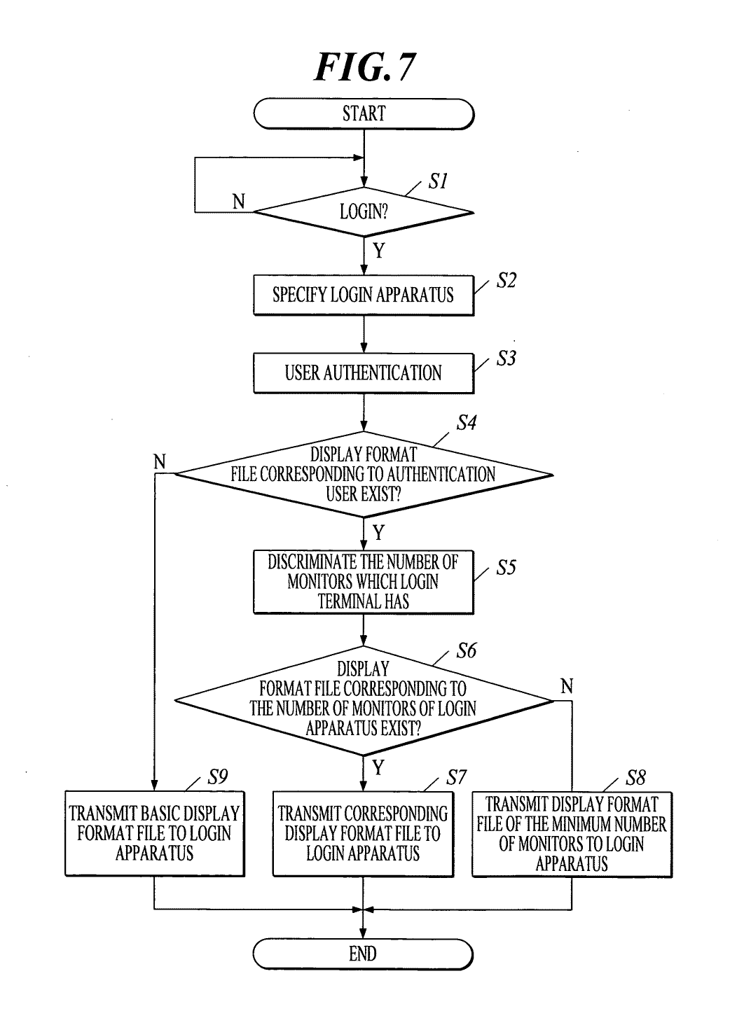

First, in view of FIG. 7, the display format file providing processes performed in the server 3 when the doctor logged in to the medical image display apparatus 5A or 5B will be described.

In the medical image display apparatus 5A or 5B, when the login operation is performed via the operation section 52 by the doctor, the login information of the user name (or user ID) and password inputted in the login operation is transmitted to the server 3. At this time, information of the host name of the medical image display apparatuses 5A and 5B to which login is performed is transmitted together with the login information.

Hereinafter, the following explanation will be described on condition that the login operation is performed in the medical image display apparatus 5A.

As shown in FIG. 7, in the server 3, the control section 31 receives the login information from any of the medical image display apparatus 5A or 5B, it is discriminated that the login operation is performed (step S1; Y), the medical image display apparatus by which the login is performed is identified by control section 31 based on information of the host name received together with the login information (step S2). Specifically, the identification is performed by matching the received host name and the host name registered in the monitor file 343. Hereinafter, the medical image display apparatus by which the login is performed is often referred to as login apparatus.

Next, in the control section 31, the login information and the registration user file 341 are matched, and the user authentication is performed (step S3). Here, when the user name and password corresponding to the login information are not retrieved in the registration user file 341, the error information for informing about error of the user authentication is transmitted to the medical image display apparatus 5A via the communication section 32.

When the user authentication completed, in the control section 31, existence or nonexistence of the display format file corresponding to the authentication user is discriminated based on the registration user file 341 (step S4). When it is discriminated that the file name of the display format file corresponding to the authentication user in the registration user file 341 is registered and the display format file exists (step S4; Y), the process proceeds to the process of step S5. Also, when it is discriminated that the display format file corresponding to the authentication user does not exist (step S4; N), the process proceeds to the process of step S9.

First, the following explanation will be described on condition that it is discriminated that the display format file exists.

In this case, in the control section 31, the number of monitors of the medical image display apparatus 5A identified as the login apparatus is discriminated in view of the monitor file 343 (step S5). In the monitor file 343 shown in FIG. 5, since monitor IDs corresponding to the host name “DocA” of the medical image display apparatus 5A are registered as three monitors “01”, “02” and “03”, it is discriminated that the number of monitors is three.

After that, in the control section 31, it is discriminated on the basis of the display format table 342 whether the display format file corresponding to the number of monitors of the medical image display apparatus 5A exists or not in the display format file corresponding to the authentication user (step S6).

Since the medical image display apparatus 5A has three monitors, it is discriminated the file “X3” in which the number of monitors is three is matched in the display format file “X1”, “X2”, “X3” of the authentication user “X”.

When the display format file in which the number of monitors is matched exists (step S6; Y), the matched display format file is read out from the display format table 342 by the control section 31 and transmitted to the medical image display apparatus 5A via the communication section 32 (step S7), and this process is ended.

On the other hand, when the matched display format file does not exist (step S6; N), the display format file of the minimum number of monitors in the display format files registered about the authentication user is transmitted to the medical image display apparatus 5A (step S8), and this process is ended.

For example, with respect to the authentication user “X”, when the display format file “X3” is not registered and only “X1” and “X2” are registered, the display format file “X1” registered as the minimum number of monitors “1” is transmitted to the medical image display apparatus 5A. that is, when the display format file of the number of monitors more than the number of monitors of the medical image display apparatus 5A is adapted with respect to the medical image display apparatus 5A, the object data to be displayed in nonexistent monitor is displayed together with the object data to be displayed in other monitor in an overlap status. In order to avoid this overlap display, the display format file for displaying by the monitor configuration always less than the number of monitors of the medical image display apparatus 5A is selected. In addition, it may be also adapted to select arbitrary one from among the display format file of the number of monitors less than the number of monitors of the medical image display apparatus 5A.

Next, in step S4, the following explanation will be described on condition that the display format file corresponding to the authentication user does not exist.

In this case, the basic format file is transmitted to the medical image display apparatus 5A (step S9), this process is ended.

According to this, when the display format file is transmitted to the medical image display apparatus 5A, the display of the object data is performed according to this display format file in the medical image display apparatus 5A.

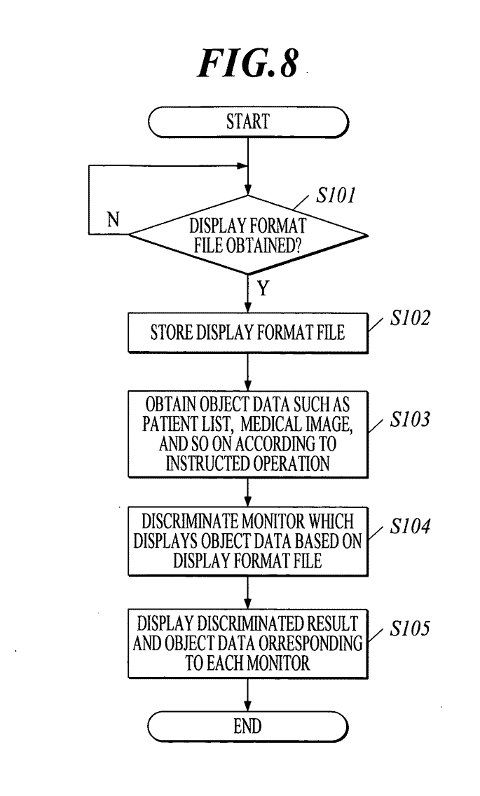

In view of FIG. 8, the object data display processes performed in the medical image display apparatus 5A will be described. Here, in addition, the process operation of the medical image display apparatus 5A will be described. Also, the same process is performed in the medical image display apparatus 5B.

As shown in FIG. 8, when the display format file is obtained from the server 3 (step S101; Y), the display format file is stored in the RAM 55 (step S102). Then, when instruction operations for displaying patient list, medical images, and so on are performed by the doctor, the object data is obtained by the control of the control section 51 (step S103). At the time of the obtaining, the request information generated by the control section 51 is transmitted to the server 3. Since the control of the NAS 4 is performed by the server 3 so as to transfer the object data designated according to the request information, the medical image display apparatus 5A obtains the object data from the NAS 4.

In the control section 51, the monitor for displaying the object data is discriminated based on the obtained display format file (step S104). When the display format file “X3” shown in FIG. 4 is obtained, the “patient list screen” is set in the monitor of the monitor ID “01”, the “radiogram interpretation screen” of the single format is set in the monitor of the monitor ID “02”, and the “report screen” is set in the monitor of monitor ID “03”. Since the patient list is obtained from the server 3 and each object data of the medical image and report is obtained from the NAS 4, in the medical image display apparatus 5A, the object data of the patient list, the medical image and the object data of report are discriminated as monitor 53a of monitor ID “01”, monitor 53b of monitor ID “02” by the single format and monitor 53c of monitor ID “03” respectively.

Then, according to a discriminating result, when the object data corresponding to each of monitor 53a-53c is displayed (step S105), this process is ended.

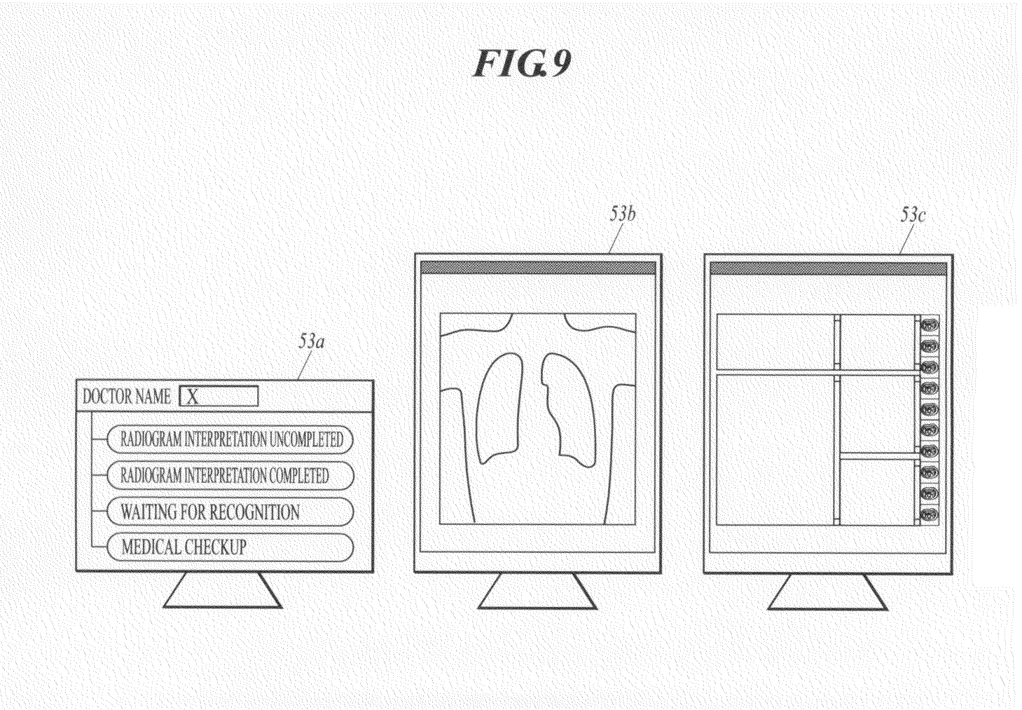

FIG. 9 shows that display example.

As shown in FIG. 9, the patient list screen is displayed on the monitor 53a, the radiogram interpretation screen of the single format is displayed on the format monitor 53b, and the report screen is displayed on the monitor 53c. Each object data is displayed by the display format according to the display format file.

As mentioned above, according to this embodiment, the display format file corresponding to the monitor configuration of the medical image display apparatuses 5A and 5B is prepared and registered in advance in the server 3 for each user. According to this, at the time of authentication of login, the authentication user and the display format file corresponding to the monitor configuration of the medical image display apparatuses 5A and 5B to which login is performed can be obtained, and it is possible to display the object data based on this.

Consequently, when the doctor login to any one of the medical image display apparatuses 5A and 5B having different monitor configurations, the object data can be displayed by the display format corresponding to the monitor configuration. Since it is not necessary to perform display operations of the object data one by one according to the monitor configuration of the medical image display apparatus 5A or 5B to which login is performed, convenience is high.

Also, since display format files can be registered for each user, it is possible to not only adapt to the monitor configuration of the medical image display apparatuses 5A and 5B by preparing the display format file based on the display contents corresponding to the choice or the style of the doctor, but customize the display contents by the doctor.

Also, since the display format file is stored so that each monitor ID and the display contents in the monitor correspond, it is possible to set which monitor displays what object data. Also, the layouts, and so on as display contents can be registered with respect to medical images, and it is possible to customize the display format in detail.

In addition, the embodiment is not limited to this because of the suitable examples to which the present invention is adapted.

For example, it may be allowable to provide the display format file for each group such as internal medicine, surgery, and so on. In this case, the display format file to be used for each group can be utilized in common, and it is possible to unify the work procedure of doctors.

Also, without providing the display format file for each user as the above mentioned embodiment, it may be allowable to register a display format for each monitor configuration capable of being used by each of the medical image display apparatuses 5A and 5B as a common display format file for each of medical image display apparatuses 5A and 5B. For example, when the maximum number of monitors in all medical image display apparatuses 5A and 5B is three, three display format files corresponding to the number 1, 2 and 3 of monitors are registered. It is possible to adapt to all monitor configurations for any user by using these display format files.

### INDUSTRIAL APPLICABILITY

It is possible to utilize it in the field of the display technology, and adapt to medical image systems for displaying medical image data and display object data relating to other medical images on monitors.

1. A medical image system including a plurality of medical image display apparatuses each equipped with one or more monitors, comprising:
a storage section for storing a display format of object data corresponding to monitor configuration in each of the plurality of medical image display apparatuses;
a discriminating section for discriminating the monitor configuration in the medical image display apparatus that displays the object data from among the plurality of medical image display apparatuses; and
a display control section that causes a monitor of the medical image display apparatus display the object data based on the display format corresponding to the discriminated monitor configuration from among the stored display formats.

2. The medical image system according to claim 1, wherein
said storage section stores the display format corresponding to the monitor configuration for each user,
said discriminating section discriminates the monitor configuration of the medical image display apparatus that displays the object data and a login user to the medical image display apparatus, and
said display control section causes the object data to be displayed on the medical image display apparatus based on the display format corresponding to the discriminated monitor configuration and the display format corresponding to the user.

3. The medical image system according to claim 1, wherein the object data includes the medical image data.

4. The medical image system according to claim 1, wherein the display format includes a format for setting a type of the object data to be displayed on each monitor.

5. The medical image system according to claim 1, wherein said display control section causes the object data to be displayed on the monitor of the medical image display apparatus based on the display format corresponding to the monitor configuration of the minimum number of monitors from among the display formats stored in said storage section when the display format corresponding to the discriminated monitor configuration is not stored in said storage section.
